(19) United States
(12) Patent Application Publication
(10) Pub. No.: US 2013/0129104 A1
(43) Pub. Date: May 23, 2013

(54) SYSTEM AND METHOD FOR ACOUSTIC NOISE MITIGATION IN A COMPUTED TOMOGRAPHY SCANNER

(71) Applicants: Ashutosh Joshi, Waukesha, WI (US); Joseph James Lacey, Cambridge, WI (US)

(72) Inventors: Ashutosh Joshi, Waukesha, WI (US); Joseph James Lacey, Cambridge, WI (US)

(21) Appl. No.: 13/298,666

(22) Filed: Nov. 17, 2011

(51) Int. Cl. G10K 11/16; A61B 6/03

(52) U.S. Cl. 381/71.3; 378/4

(57) ABSTRACT

A CT system is provided that includes an outer housing, a rotatable gantry positioned within the outer housing and having a gantry opening to receive an object to be scanned, an x-ray source mounted on the rotatable gantry and configured to project an x-ray beam toward the object, and a detector array mounted on the rotatable gantry and configured to detect x-ray energy passing through the object and generate a detector output responsive thereto that can be reconstructed into an image of the object. A hybrid noise mitigation system is included in the CT system that is configured to mitigate noise generated by the CT system during operation, the hybrid noise mitigation system comprising a passive noise mitigation device configured to control noise in a passive manner and an active noise mitigation device configured to control noise in an active manner.

### BACKGROUND OF THE INVENTION

Embodiments of the invention relate generally to a computed tomography (CT) scanner and, more particularly, to a system and method for mitigating acoustic noise in a CT scanner.

Typically, in CT imaging systems, an x-ray source emits a fan-shaped beam toward a subject or object, such as a patient or a piece of luggage. Hereinafter, the terms “subject” and “object” shall include anything capable of being imaged. The beam, after being attenuated by the subject, impinges upon an array of radiation detectors. The intensity of the attenuated beam radiation received at the detector array is typically dependent upon the attenuation of the x-ray beam by the subject. Each detector element of the detector array produces a separate electrical signal indicative of the attenuated beam received by each detector element. The electrical signals are transmitted to a data processing system for analysis which ultimately produces an image.

Generally, the x-ray source and the detector array are rotated about the gantry within an imaging plane and around the subject. X-ray sources typically include x-ray tubes, which emit the x-ray beam at a focal point. X-ray detectors typically include a collimator for collimating x-ray beams received at the detector, a scintillator for converting x-rays to light energy adjacent the collimator, and photodiodes for receiving the light energy from the adjacent scintillator and producing electrical signals therefrom. Typically, each scintillator of a scintillator array converts x-rays to light energy. Each scintillator discharges light energy to a photodiode adjacent thereto. Each photodiode detects the light energy and generates a corresponding electrical signal. The outputs of the photodiodes are then transmitted to the data processing system for image reconstruction.

In operation, CT scanners generate acoustic noise from a variety of sources. For example, cooling fans for various sub-systems, cooling pumps, the x-ray tube rotor, gantry bearings, gantry fans, and so forth, may all generate acoustic noise. Additionally, rotation of the gantry also produces acoustic noise, and it is recognized that such noise from gantry rotation will only increase in future generation CT systems based on the increased speed gantry rotation, and accompanying increased aero-acoustic noise generated therefrom, found therein. While the production of noise from these sources does not directly affect the medical imaging process, the noise may be uncomfortable or disconcerting to an imaging subject. This is especially true for CT systems having an air cooled gantry, where the acoustic noise is increased based on the use of fans to cool the gantry using scan room air.

In some prior art CT systems, the issue of noise has been ignored, with no noise reduction methods or systems being employed to reduce noise generated by the CT system. In other prior art CT systems, “noise cancellation” devices have been developed in an attempt to reduce the imaging subject's perception of the noise and thereby present a more comfortable environment for the subject during the imaging process. However, prior noise cancellation devices and methods have not met general acceptance for a number of reasons. For example, for some prior art CT systems having an air cooled gantry, noise mitigation has been achieved by derating cooling fans in the system. However, such derating of the cooling fans is generally still insufficient to completely address the noise problem. To address the issue of noise, other prior art CT systems have employed a chilled gantry that is closed/sealed to the external environment. While such a chilled gantry cooling system construction is effective in cooling the CT system and reducing the level of acoustic noise to the environment, the chilled gantry is extremely expensive to construct and operate, as large and expensive heat exchangers are required to provide adequate cooling for the CT system.

Therefore, it would be desirable to design an apparatus and method for mitigating noise in a CT scanner.

### BRIEF DESCRIPTION OF THE INVENTION

Embodiments of the invention include a directed method and system for mitigating acoustic noise in a CT scanner.

In accordance with one aspect of the invention, a CT system includes an outer housing, a rotatable gantry positioned within the outer housing and having a gantry opening to receive an object to be scanned, an x-ray source mounted on the rotatable gantry and configured to project an x-ray beam toward the object, a detector array mounted on the rotatable gantry and configured to detect x-ray energy passing through the object and generate a detector output responsive thereto that can be reconstructed into an image of the object, and a hybrid noise mitigation system configured to mitigate noise generated by the CT system during operation, the hybrid noise mitigation system comprising a passive noise mitigation device configured to control noise in a passive manner and an active noise mitigation device configured to control noise in an active manner.

In accordance with another aspect of the invention, a CT system includes a rotatable gantry having a gantry opening to receive an object to be scanned and an outer housing positioned about the rotatable gantry, with the outer housing having gantry inlet ducts and gantry exhaust ducts formed therein each including a fan for transferring air into and out of an interior of the outer housing, respectively. The CT system also includes an x-ray source mounted on the rotatable gantry and configured to project an x-ray beam toward the object, a detector array mounted on the rotatable gantry and configured to detect x-ray energy passing through the object and generate a detector output responsive thereto that can be reconstructed into an image of the object, and a heat exchanger corresponding to each of the x-ray source and the detector array and mounted on the rotatable gantry, the heat exchangers configured to provide cooling to the x-ray source and the detector array. The CT system further includes a plurality of noise mitigation devices configured to mitigate noise generated by the CT system during operation thereof, wherein a noise mitigation device is provided for each of the gantry inlet ducts, gantry exhaust ducts, and heat exchangers to mitigate noise produced thereby in at least one of a passive manner and an active manner.

In accordance with yet another aspect of the invention, a method for mitigating noise in a CT system includes integrating a plurality of noise mitigation devices into existing components and features of the CT system, passively reducing the level of audible acoustic noise generated by the CT system by way of the plurality of noise mitigation devices, and actively reducing the level of audible acoustic noise generated by the CT system by way of the plurality of noise mitigation devices. The plurality of noise mitigation devices are configured to reduce the level of audible acoustic noise generated by at least one of CT gantry rotation, gantry fans, x-ray tube operation, x-ray tube heat exchanger fans, and x-ray detector heat exchanger fans.

Various other features and advantages will be made apparent from the following detailed description and the drawings.

### BRIEF DESCRIPTION OF THE DRAWINGS

The drawings illustrate preferred embodiments presently contemplated for carrying out the invention.

In the drawings:

FIG. 1 is a pictorial view of a CT imaging system.

FIG. 2 is a block schematic diagram of the system illustrated in FIG. 1.

FIG. 3 is a block schematic diagram of the system illustrated in FIG. 1, illustrating noise sources that generate noise during operation of the CT imaging system.

FIG. 4 is a schematic diagram of a noise mitigation device incorporated into a heat exchanger of the CT system of FIG. 1 according to an embodiment of the invention.

FIG. 5 is a schematic diagram of a noise mitigation device incorporated into a heat exchanger of the CT system of FIG. 1 according to another embodiment of the invention.

FIG. 6 is a schematic diagram of a noise mitigation device incorporated into a gantry exhaust duct of the CT system of FIG. 1 according to an embodiment of the invention.

FIG. 7 is a schematic diagram of a noise mitigation device incorporated into a gantry inlet duct of the CT system of FIG. 1 according to an embodiment of the invention.

FIG. 8 is a block schematic diagram of a CT imaging system having a system level noise controller for controlling noise sources that generate noise during operation of the CT imaging system according to an embodiment of the invention.

FIG. 9 is a pictorial view of a CT system for use with a non-invasive package inspection system according to an embodiment of the invention.

### DETAILED DESCRIPTION OF THE PREFERRED EMBODIMENT

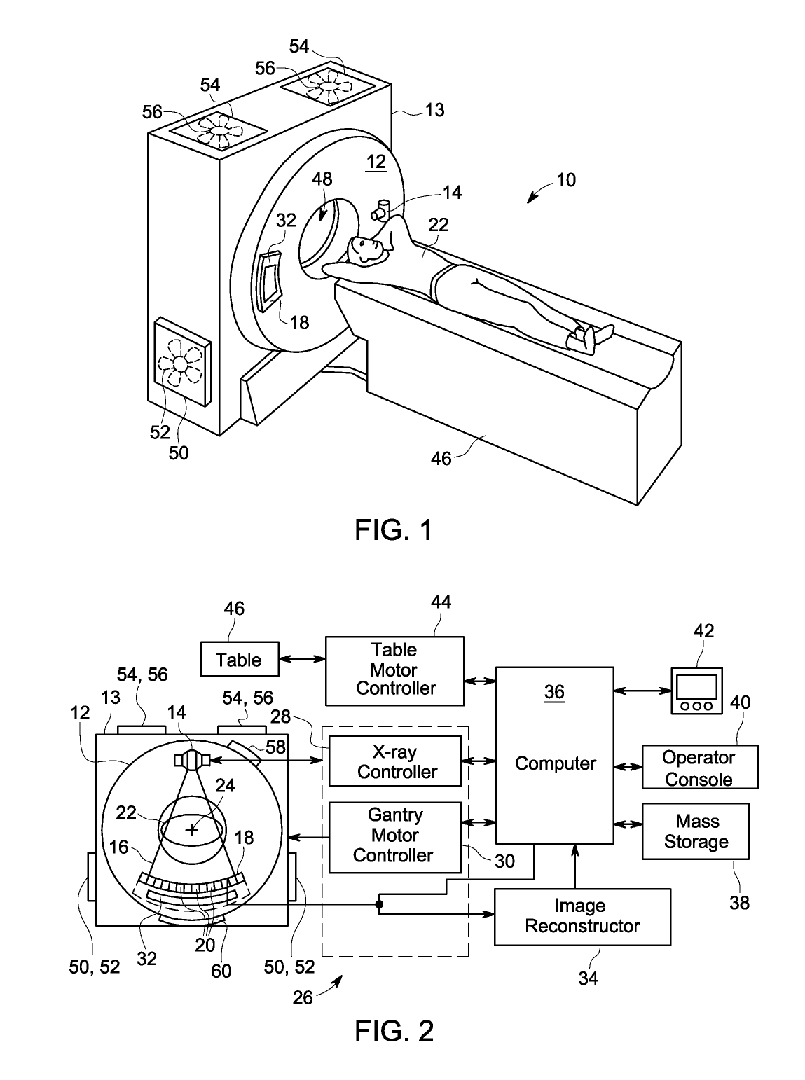

Referring to FIG. 1, a computed tomography (CT) imaging system 10 is shown as including a rotatable gantry 12 representative of a “third generation” CT scanner. An outer housing 13 is positioned about the gantry 12 so as to substantially enclose the gantry. Gantry 12 has an x-ray source 14 that projects a beam of x-rays toward a detector assembly or collimator 18 on the opposite side of the gantry 12. Referring now to FIG. 2, detector assembly 18 is formed by a plurality of detectors 20 and data acquisition systems (DAS) 32. The plurality of detectors 20 sense the projected x-rays 16 that pass through a medical patient 22, and DAS 32 converts the data to digital signals for subsequent processing. Each detector 20 produces an analog electrical signal that represents the intensity of an impinging x-ray beam and hence the attenuated beam as it passes through the patient 22. During a scan to acquire x-ray projection data, gantry 12 and the components mounted thereon rotate about a center of rotation 24.

Rotation of gantry 12 and the operation of x-ray source 14 are governed by a control mechanism 26 of CT system 10. Control mechanism 26 includes an x-ray controller 28 that provides power and timing signals to an x-ray source 14 and a gantry motor controller 30 that controls the rotational speed and position of gantry 12. An image reconstructor 34 receives sampled and digitized x-ray data from DAS 32 and performs high speed reconstruction. The reconstructed image is applied as an input to a computer 36 which stores the image in a mass storage device 38.

Computer 36 also receives commands and scanning parameters from an operator via console 40 that has some form of operator interface, such as a keyboard, mouse, voice activated controller, or any other suitable input apparatus. An associated display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 and gantry 12. Particularly, table 46 moves patients 22 through a gantry opening 48 of FIG. 1 in whole or in part.

As further shown in FIGS. 1 & 2, CT system 10 also includes a plurality of cooling systems or components that function to provide an acceptable temperature and operating environment for the CT system 10 and prevent overheating of the CT system 10 and specific components thereof during operation. As shown in FIG. 1, the CT system 10 is configured such that the gantry 12 of CT system 10 is air cooled. Gantry inlet ducts 50 are provided on outer housing 13 of the CT system 10, with fans 52 included in the gantry inlet ducts 50 to draw air from the ambient environment into the housing 13 of the CT system 10 and into contact with the rotating gantry 12 so as to provide cooling thereto. Gantry exhaust ducts 54 are also provided on housing 13, with fans 56 included in the gantry exhaust ducts 54 to force air that has become heated from contact with the gantry 12 out from within the housing 13 and into the ambient environment. As shown in FIG. 2, heat exchangers 58, 60 are also included in CT system 10 for cooling the x-ray source 14 and the x-ray detector array 18, respectively, with the heat exchangers 58, 60 being mounted on gantry 12 so as to rotate thereon. According to an embodiment of the invention, heat exchangers 58, 60 have a similar construction and are configured as liquid-air heat exchangers that pump cooling fluid to x-ray source 14 and detector array 18 so as to draw heat from the x-ray source 14 and detector array 18 and reduce the operating temperature thereof.

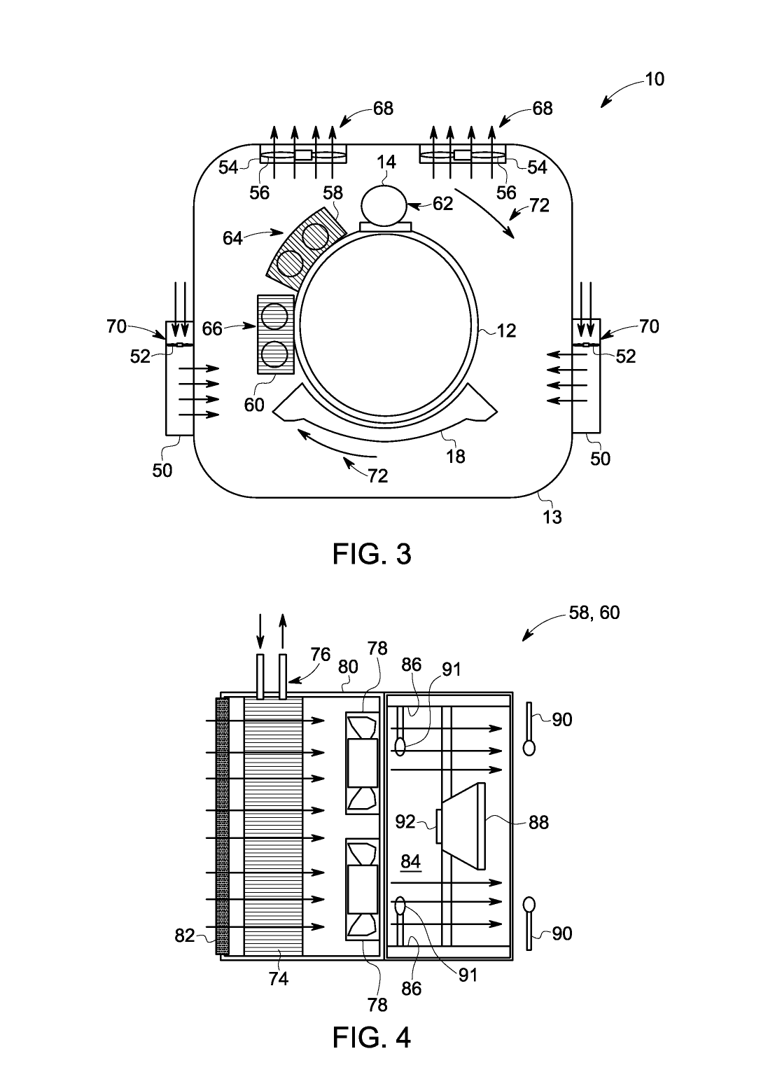

In operation, CT system 10 generates acoustic noise from a variety of sources. For example, cooling fans for various sub-systems, cooling pumps, the x-ray tube rotor, gantry bearing, gantry fans, and so forth, may all generate acoustic noise. Such noise sources are generally indicated in FIG. 3, with noise from x-ray source 14 (i.e., x-ray tube rotor) indicated as 62, noise from the x-ray source heat exchanger 58 indicated as 64, noise from the x-ray detector heat exchanger indicated as 66, noise from the gantry exhaust duct fans 56 indicated as 68, noise from the gantry inlet duct fans 52 indicated as 70, and noise from the rotation of the gantry 12 indicated as 72.

Referring now to FIGS. 4-7, noise mitigation components/devices incorporated into CT system 10 for reducing the level of audible acoustic noise are shown according to various embodiments of the invention. According to embodiments of the invention, passive noise mitigation devices/methods, active noise mitigation devices/methods, and/or hybrid passive-active noise mitigation methods may be employed at the device level and at the CT system level to control the level of noise that is projected to the gantry opening 48 (FIG. 1) of the CT system 10 and to the surrounding external environment.

Referring now to FIG. 4, a detailed view of the x-ray detector and x-ray source heat exchangers 58, 60 is provided according to one embodiment of the invention, with noise mitigation features incorporated therein. As mentioned above, according to one embodiment, the structure of tube heat exchanger 58 and detector heat exchanger 60 is similar/identical, and thus FIG. 4 is illustrative of both heat exchangers. The heat exchanger 58, 60 includes a cooling unit 74 and arrangement of tubing 76 that circulates a cooling fluid therethrough. Chilled cooling fluid is pumped from cooling unit 74 and through tubing 76 of liquid-air heat exchanger 58, 60 to x-ray source 14 or detector array 18 so as to remove heat therefrom, with heated fluid then being returned to the heat exchanger 58, 60. A plurality of fans 78 are included in heat exchanger 58, 60 and are positioned adjacent the cooling unit 74 in a fan plenum 80 to aid in removing heat from the cooling fluid. According to the embodiment of FIG. 4, fans 78 operate in a “pull” mode to draw heated air that is in proximity to cooling unit 74 away therefrom. More specifically, air is drawn into fan plenum 80 through an air filter 82, passes over cooling unit 74 so as to be heated thereby, and is then drawn away via the “pulling” of air induced by fans 78. The heated air that is pulled away by fans 78 is then blown out through an outlet duct 84 of heat exchanger 58, 60, with the air then subsequently being expelled from CT system 10 by way of gantry exhaust fans 56 (FIGS. 1-3).

As shown in FIG. 4, the heat exchanger 58, 60 is configured to “passively” mitigate noise generated by the fans 78 included therein. For providing such passive noise mitigation, a layer of foam 86 is positioned within duct 84 to reduce the level of audible acoustic noise generated by fans 78 of heat exchanger 58, 60. The foam layer 86 is configured to mitigate the noise generated by fans 78 by reducing the high frequency component of the noise. According to embodiments of the invention, the foam layer 86 may be formed of a suitable acoustic foam material, such as polyurethane or another suitable polymer composite, with the layer further having a desired profile, such as a convoluted pattern (i.e., egg-crate pattern), wedge pattern, pyramidal pattern, or other suitable profile. Additionally, it is recognized that noise generated from other noise sources, such as pumps (not shown) and the rotor of x-ray tube 14 (FIG. 1), may be damped using passive vibration isolation and/or by appropriately mounting such components to the super-structure of CT system 10.

According to another embodiment of the invention, the heat exchanger 58, 60 is configured to using a “hybrid” type noise mitigation configuration. That is, in addition to the passive noise mitigation provided by foam layer 86, the heat exchanger 58, 60 is further configured to apply “active” noise mitigation for the noise generated by the fans 78. In one embodiment, such active noise cancellation is used when the level of noise generated by the CT system rises above a minimum noise threshold. Such a noise threshold may be crossed when the CT system is operating on high power and in a hot scan room environment, while the noise threshold may not be crossed when the CT system is operating on low power and in a cold scan room environment.

As shown in FIG. 4, a speaker (or arrangement of speakers) 88 is positioned within outlet duct 84 that provides for active noise mitigation. The speaker 88 is configured to generate sound at the same frequency as fans 78, but that is out of phase with the noise generated by fans 78. The out of phase sound generated by speaker(s) 88, at the same frequency as the fan noise, thus functions to cancel out the noise generated by fans 78, thereby reducing the level of audible acoustic noise generated by fans 78 of heat exchanger 58, 60.

In order to determine the frequency of acoustic noise generated by the fans 78, one or more microphones 90, 91 are provided to measure/record the fan noise. In one embodiment of the invention, only reference microphones 91 are employed for purposes of determining a frequency at which sound is to be generated by speaker 88, according to a feed-forward active noise mitigation technique. Reference microphones 91 are positioned within outlet duct 84 to measure/record the fan noise, with the fan noise measured/recorded by reference microphones 91 being output/provided to a controller 92 having a digital signal processing (DSP) algorithm stored thereon. The controller 92 receives the output from reference microphones 91 and inputs it to the DSP algorithm in order to determine a proper frequency and phase at which noise should be generated by speaker(s) 88, according to the feed-forward technique.

In another embodiment, both reference microphones 91 and error microphones 90 are employed for purposes of determining a frequency at which sound is to be generated by speaker 88, according to a feedback active noise mitigation technique. Reference microphones 91 are positioned within outlet duct 84 to measure/record the fan noise, with error microphones 90 being positioned adjacent outlet duct 84 to further minimize the acoustic noise. That is, the fan noise measured/recorded by reference microphones 91 is output/provided to controller 92 having the digital signal processing (DSP) algorithm stored thereon, with the controller 92 receiving the output from reference microphones 91 and inputting it to the DSP algorithm in order to determine a proper frequency and phase at which noise should be generated by speaker(s) 88. The speaker(s) then generate sound at the same frequency as noise generated by fans 78 but that is out of phase therewith, so as to mitigate/cancel the fan noise. The error microphones 90 measure/record any acoustic noise that might still be present after a noise cancellation between the fan noise and speaker sound, to determine if further adjustment of the sound generated by speaker(s) 88 is needed. An output may thus be generated by error microphones 90 and provided to controller 92 for input to the DSP algorithm in order to determine an adjustment to the frequency and phase at which noise should be generated by speaker(s) 88. Thus, by controlling operation of speaker 88 by way of the DSP algorithm of controller 92, the speaker 88 is able to actively control noise at a plurality of different fan speeds.

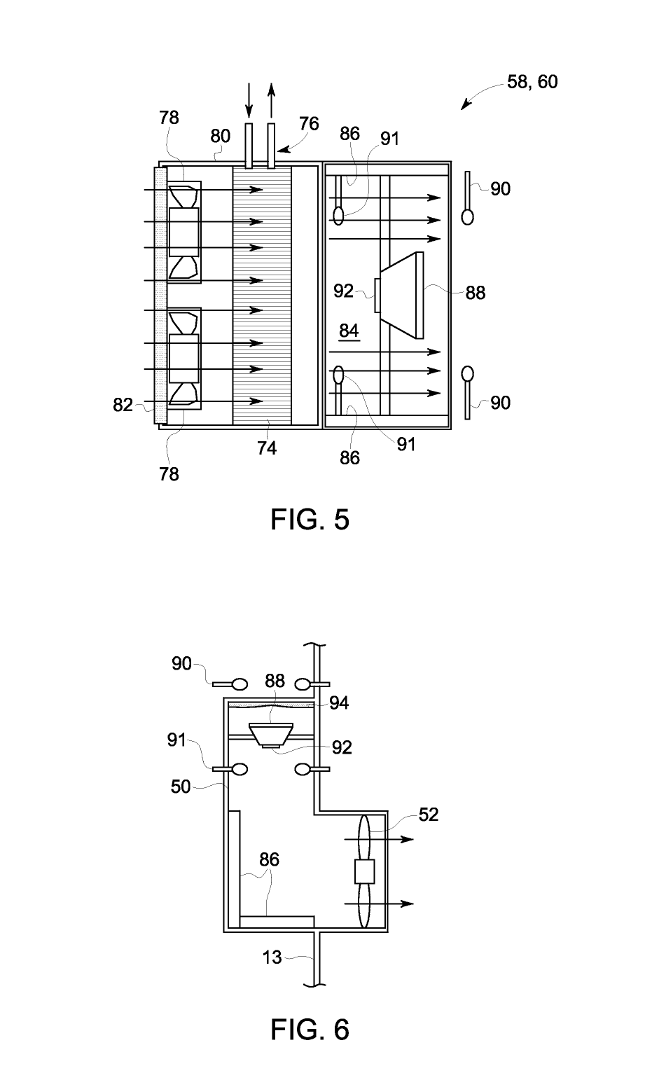

Referring now to FIG. 5, a detailed view of heat exchanger 58, 60 (i.e., both detector and tube heat exchangers) is provided according to another embodiment of the invention. The configuration of heat exchanger 58, 60 is similar to that shown in FIG. 4, except that the fans 78 included in the heat exchanger 58, 60 operate in a “push” mode to blow air across the cooling unit 74. In operation of heat exchanger 58, 60, air is drawn into fan plenum 80 through an air filter 82, and air is “pushed” by fans 78 so as flow/pass over the cooling unit 74 so as remove heat from the cooling fluid. The air flow is pushed past cooling unit 74 and is blown out through outlet duct 84 of heat exchanger 58, 60, with the air then subsequently being expelled from CT system 10 by way of exhaust fans 56 (FIGS. 1-3).

As shown in FIG. 5, the heat exchanger 58, 60 is configured to “passively” mitigate noise generated by fans 78 via a foam layer 86 positioned within outlet duct 84. The foam layer 86 is configured to mitigate the noise generated by fans 78 by reducing the high frequency component of the noise, such the level of audible acoustic noise generated by fans 78 of heat exchanger 58, 60 is reduced. The foam layer 86 may be formed of any suitable acoustic foam material, such as polyurethane or another suitable polymer composite, and may have any suitable profile or pattern, such as a convoluted pattern (i.e., egg-crate pattern), wedge pattern, or pyramidal pattern, for example.

According to another embodiment of the invention, and as shown in phantom in FIG. 5, the heat exchanger 58, 60 includes a speaker (or arrangement of speakers) 88 positioned within outlet duct 84 that provides for “active” noise mitigation. The speaker 88 is configured to generate sound at the same frequency as fans 78, but that is out of phase with the noise generated by fans 78. The out of phase sound generated by speaker(s) 88, at the same frequency as the fan noise, thus functions to cancel out the noise generated by fans 78, thereby actively reducing the level of audible acoustic noise generated by fans 78 of heat exchanger 58, 60. In order to determine the frequency of noise generated by the fans 78, one or more microphones 90, 91 are positioned adjacent outlet duct 84 to measure/record the fan noise. The fan noise measured/recorded by microphones 90 is provided to a digital signal processing (DSP) algorithm stored in controller 92 in order to determine a proper frequency and phase at which noise should be generated by speaker(s) 88. According to one embodiment of the invention, only reference microphone 91 are employed to provide input to controller 92 for purposes of determining a frequency at which sound is to be generated by speaker 88, according to a feed-forward active noise mitigation technique. According to another embodiment of the invention, both reference microphones 91 and error microphones 90 are employed to provide input to controller 92 for purposes of determining a frequency at which sound is to be generated by speaker 88, according to a feedback active noise mitigation technique. By controlling operation of speaker 88 by way of the DSP algorithm in controller 92, the speaker(s) 88 is able to actively control noise at a plurality of different fan speeds. Thus, heat exchanger 58, 60 employs a “hybrid” method/structure for noise mitigation. That is, in addition to the passive noise mitigation provided by foam layer 86, the speaker(s) 88 provides “active” noise mitigation for the noise generated by the fans 78.

Referring now to FIG. 6, a detailed view of gantry inlet duct 50 of CT system 10 is shown, with noise mitigation features incorporated therein. The gantry inlet duct 50 is formed in the housing 13 of the CT system 10 and includes a fan 52 positioned therein to draw/pull air from the outside ambient environment, into the interior of housing 13 of the CT system 10, and into contact with the rotating gantry 12 so as to provide cooling thereto. Air is drawn through an air filter 94 and into gantry inlet duct 50 by way of fan 52, with the air being directed into housing 13 so as to cool the rotating gantry 12 of CT system 10.

Included in gantry inlet duct 50 is a layer of foam 86 configured to reduce the level of audible acoustic noise generated by fan 52. The foam layer 86 is formed of an acoustic foam material (e.g., polyurethane or another suitable polymer composite), so as to mitigate the noise generated by fan 52 by reducing the high frequency content of the noise. The foam layer 86 thus functions as a passive method/device for noise mitigation of the fan 52 in gantry inlet duct 50.

According to one embodiment of the invention, a speaker (or arrangement of speakers) 88 is positioned within gantry inlet duct 50 that provides for active noise mitigation. The speaker 88 is configured to generate sound at the same frequency as fan 52, but that is out of phase with the noise. The out of phase sound generated by speaker 88, at the same frequency as the fan noise, thus functions to cancel out the noise generated by fan 52, thereby actively reducing the level of audible acoustic noise generated by fan 52 in gantry inlet duct 50. In order to determine the frequency of noise generated by fan 52, one or more microphones 90, 91 are positioned to measure/record the fan noise. The fan noise measured/recorded by microphones 90 is provided to a digital signal processing (DSP) algorithm in controller 92 in order to determine a proper frequency and phase at which noise should be generated by speaker 88. According to one embodiment of the invention, only reference microphone 91 are employed to provide input to controller 92 for purposes of determining a frequency at which sound is to be generated by speaker 88, according to a feed-forward active noise mitigation technique. According to another embodiment of the invention, both reference microphones 91 and error microphones 90 are employed to provide input to controller 92 for purposes of determining a frequency at which sound is to be generated by speaker 88, according to a feedback active noise mitigation technique. By controlling operation of speaker 88 by way of the DSP algorithm, the speaker 88 is able to actively control noise at a plurality of different fan speeds. Thus, according to one embodiment, gantry inlet duct 50 includes and employs a “hybrid” method/structure for noise mitigation. That is, in addition to the passive noise mitigation provided by foam layer 86, the speaker(s) 88 provides “active” noise mitigation for the noise generated by fan 52 in gantry inlet duct 50.

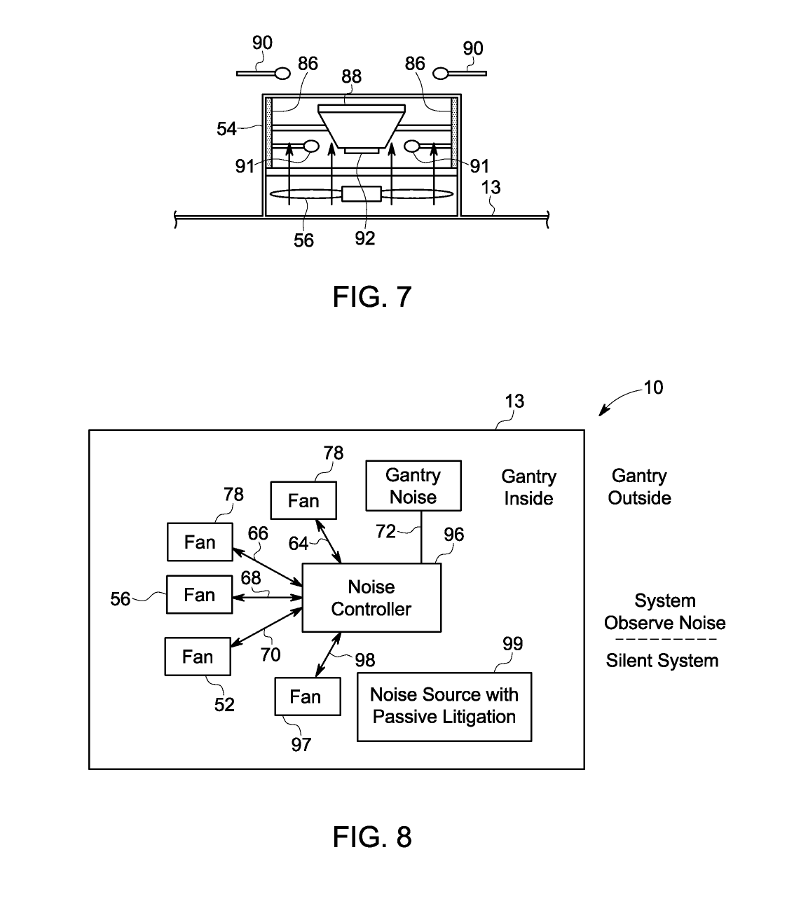

Referring now to FIG. 7, a detailed view of gantry exhaust duct 54 of CT system 10 is shown, with noise mitigation features incorporated therein. The gantry exhaust duct 54 is formed in the housing 13 of the CT system 10 and includes a fan 56 positioned therein to push air from the interior of housing 13 of the CT system 10 out to the outside ambient environment, so as to remove air that has become heated from contact with the gantry 12 out from the CT system 10. Air from the interior of CT system 10 is drawn into gantry exhaust duct 54 by way of fan 56 and subsequently pushed out into the ambient environment.

Included in gantry exhaust duct 54 is a layer of foam 86 configured to reduce the level of audible acoustic noise generated by fan 56. The foam layer 86 is formed of an acoustic foam material (e.g., polyurethane or another suitable polymer composite), so as to mitigate the noise generated by fan 56 by reducing the high frequency content of the noise. The foam layer 86 thus functions as a passive method/device for noise mitigation of the fan 56 in gantry exhaust duct 54.

According to one embodiment of the invention, and as shown in phantom in FIG. 7, a speaker (or arrangement of speakers) 88 is positioned within gantry exhaust duct 54 that provides for active noise mitigation. The speaker 88 is configured to generate sound at the same frequency as fan 56, but that is out of phase with the noise. The out of phase sound generated by speaker 88, at the same frequency as the fan noise, thus functions to cancel out the noise generated by fan 56, thereby actively reducing the level of audible acoustic noise generated by fan 56 in gantry exhaust duct 54. In order to determine the frequency of noise generated by fan 56, one or more microphones 90, 91 are positioned to measure/record the fan noise. The fan noise measured/recorded by microphones 90 is provided to a digital signal processing (DSP) algorithm in controller 92 in order to determine a proper frequency and phase at which noise should be generated by speaker 88. According to embodiments of the invention, both reference microphones 91 and error microphones 90 may be employed or only reference microphones 90 may be employed to provide input to controller 92 for purposes of determining a frequency at which sound is to be generated by speaker 88, according to feedback and feed-forward active noise mitigation techniques, respectively. By controlling operation of speaker 88 by way of the DSP algorithm, the speaker 88 is able to actively control noise at a plurality of different fan speeds. Thus, gantry exhaust duct 54 includes and employs a “hybrid” method/structure for noise mitigation. That is, in addition to the passive noise mitigation provided by foam layer 86, the speaker 88 provides “active” noise mitigation for the noise generated by fan 56 in gantry exhaust duct 54.

Referring now to FIG. 8, a block schematic diagram of the CT system 10 is shown according to another embodiment of the invention. In the embodiment of FIG. 8, CT system 10 includes a system level noise controller 96 that receives noise inputs from a plurality of sub-systems or components in the CT system 10, in order to determine an active noise mitigation scheme for minimizing acoustic noise generated by CT system 10. Such noise sources, and their associated noise inputs, can include x-ray source heat exchanger fans 78 and its noise input 64, x-ray detector heat exchanger fans 78 and its noise input 66, gantry exhaust duct fans 56 and its noise input 68, gantry inlet duct fans 52 and its noise input 70, any other cooling fans 97 included in the CT system and their noise input 98. A noise input 72 indicative of noise generated by rotation of the gantry can also be input into system level noise controller 96. The system level noise controller 96 functions to determine an ideal active noise mitigation control scheme for each respective component/sub-system (i.e., x-ray tube rotor 14, x-ray source heat exchanger 58, x-ray detector heat exchanger, gantry exhaust duct fans 56, gantry inlet duct fans 52, and rotating gantry 12) based on the associated noise inputs received therefrom, such as by inputting the noise signals into a digital signal processing (DSP) algorithm to generate a control signal that is transmitted to speaker(s) 88 included in each respective component/sub-system, with the control signal causing the speaker(s) to generate sound at proper frequency and phase that facilitates noise cancellation. It is recognized that system level noise controller 96 may be used in lieu of, or in combination with, the controllers 92 associated with each individual noise generating component/sub-system, according to embodiments of the invention.

As further illustrated in FIG. 8, passive noise mitigation is also employed to mitigate noise generated from other noise sources, such as pumps and the rotor of the x-ray tube, which are generally illustrated here as 99. Noise from such components/sub-systems may be damped using passive vibration isolation and/or by appropriately mounting such components to the super-structure of CT system 10. Thus, by way of the active noise mitigation provided and controlled by system level noise controller 96, in combination with passive noise mitigation of other components/sub-systems 99, a hybrid noise mitigation scheme is provided for CT system 10 that reduces the level of audible acoustic noise both within the gantry opening 48 (FIG. 1) of the CT system 10 and in an area surrounding the CT system 10 (i.e., outside of housing 13). A “silent” system is thus provided that is more accommodating to patients and system operators.

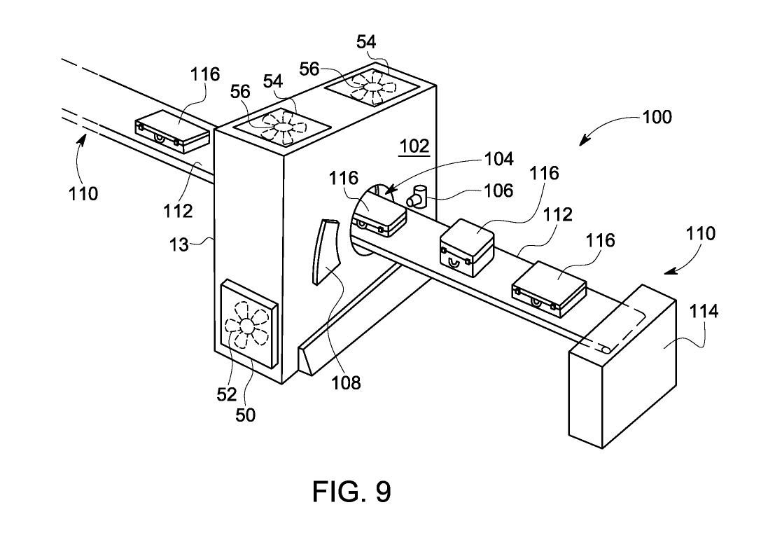

Referring now to FIG. 9, a package/baggage inspection system 100 includes a rotatable gantry 102 having an opening 104 therein through which packages or pieces of baggage may pass. The rotatable gantry 102 houses a high frequency electromagnetic energy source 106 as well as a detector assembly 108. A conveyor system 110 is also provided and includes a conveyor belt 112 supported by structure 114 to automatically and continuously pass packages or baggage pieces 116 through opening 104 to be scanned. Objects 116 are fed through opening 104 by conveyor belt 112, imaging data is then acquired, and the conveyor belt 112 removes the packages 116 from opening 104 in a controlled and continuous manner. As a result, postal inspectors, baggage handlers, and other security personnel may non-invasively inspect the contents of packages 116 for explosives, knives, guns, contraband, etc.

As shown in FIG. 9, the system 100 is configured so as to be an air cooled system. Gantry inlet ducts 50 are provided on outer housing 13 of the system 100, with fans 52 included in the gantry inlet ducts 50 to draw air from the ambient environment into the housing 13 of the system 100 and into contact with the rotating gantry 102 so as to provide cooling thereto. Gantry exhaust ducts 54 are also provided on housing 13, with fans 56 included in the gantry exhaust ducts 54 to force air that has become heated from contact with the gantry 102 out from within the housing 13 and into the ambient environment. As described in detail above, passive noise mitigation devices and active noise mitigation devices can be provided in system 100 at the device level and at the CT system level to control the level of noise that is projected to the gantry opening 104 of the system 100 and to the surrounding external environment. According to embodiments, a foam layer 86 and speakers 88, such as shown in FIGS. 4-7 can be implemented in order to passively and actively mitigate noise, respectively, so as to reduce the level of audible acoustic noise in and around system 100.

Beneficially, embodiments of the invention thus provide a system and method of noise mitigation for a CT system 10, 100. A hybrid noise mitigation scheme is provided that employs both passive and active noise control methods at both a device component level and at a system level. The hybrid noise mitigation scheme reduces the level of audible acoustic noise both within the gantry opening 48, 104 of the CT system 10, 100 (FIGS. 1, 9) and in an area surrounding the CT system 10, 100.

Therefore, according to one embodiment of the invention, a CT system includes an outer housing, a rotatable gantry positioned within the outer housing and having a gantry opening to receive an object to be scanned, an x-ray source mounted on the rotatable gantry and configured to project an x-ray beam toward the object, a detector array mounted on the rotatable gantry and configured to detect x-ray energy passing through the object and generate a detector output responsive thereto that can be reconstructed into an image of the object, and a hybrid noise mitigation system configured to mitigate noise generated by the CT system during operation, the hybrid noise mitigation system comprising a passive noise mitigation device configured to control noise in a passive manner and an active noise mitigation device configured to control noise in an active manner.

According to another embodiment of the invention, a CT system includes a rotatable gantry having a gantry opening to receive an object to be scanned and an outer housing positioned about the rotatable gantry, with the outer housing having gantry inlet ducts and gantry exhaust ducts formed therein each including a fan for transferring air into and out of an interior of the outer housing, respectively. The CT system also includes an x-ray source mounted on the rotatable gantry and configured to project an x-ray beam toward the object, a detector array mounted on the rotatable gantry and configured to detect x-ray energy passing through the object and generate a detector output responsive thereto that can be reconstructed into an image of the object, and a heat exchanger corresponding to each of the x-ray source and the detector array and mounted on the rotatable gantry, the heat exchangers configured to provide cooling to the x-ray source and the detector array. The CT system further includes a plurality of noise mitigation devices configured to mitigate noise generated by the CT system during operation thereof, wherein a noise mitigation device is provided for each of the gantry inlet ducts, gantry exhaust ducts, and heat exchangers to mitigate noise produced thereby in at least one of a passive manner and an active manner.

According to yet another embodiment of the invention, a method for mitigating noise in a CT system includes integrating a plurality of noise mitigation devices into existing components and features of the CT system, passively reducing the level of audible acoustic noise generated by the CT system by way of the plurality of noise mitigation devices, and actively reducing the level of audible acoustic noise generated by the CT system by way of the plurality of noise mitigation devices. The plurality of noise mitigation devices are configured to reduce the level of audible acoustic noise generated by at least one of CT gantry rotation, gantry fans, x-ray tube operation, x-ray tube heat exchanger fans, and x-ray detector heat exchanger fans.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A computed tomography (CT) system comprising:
an outer housing;
a rotatable gantry positioned within the outer housing and having a gantry opening to receive an object to be scanned;
an x-ray source mounted on the rotatable gantry and configured to project an x-ray beam toward the object;
a detector array mounted on the rotatable gantry and configured to detect x-ray energy passing through the object and generate a detector output responsive thereto that can be reconstructed into an image of the object; and
a hybrid noise mitigation system configured to mitigate noise generated by the CT system during operation, the hybrid noise mitigation system comprising a passive noise mitigation device configured to control noise in a passive manner and an active noise mitigation device configured to control noise in an active manner.

2. The CT system of claim 1 wherein the outer housing comprises:
an gantry inlet duct to receive ambient air from the surrounding environment into an interior volume of the outer housing to cool the CT system, the gantry inlet duct including a fan positioned therein to pull the ambient air from the surrounding environment into the interior of the outer housing; and
a gantry exhaust duct to discharge air from the interior volume of the outer housing out to the surrounding environment to cool the CT system, the gantry exhaust duct including a fan positioned therein to push air from the interior volume of the outer housing out to the surrounding environment.

3. The CT system of claim 2 wherein the passive noise mitigation device comprises a layer of acoustic foam positioned within at least one of the gantry inlet duct and the gantry exhaust duct, the layer of acoustic foam configured to reduce the high frequency content of noise generated by the fans so as to reduce the level of audible acoustic noise generated thereby.

4. The CT system of claim 2 wherein the active noise mitigation device comprises:
a speaker positioned within at least one of the gantry inlet duct and the gantry exhaust duct;
a reference microphone positioned in proximity to the at least one of the gantry inlet duct and the gantry exhaust duct to measure noise generated by the fan;
a controller configured to:
receive an output from the reference microphone indicative of the measured noise generated by the fan;
apply a digital signal processing (DSP) algorithm in order to determine a proper frequency and phase at which noise should be generated by the speaker, based on the measured noise; and
control the speaker by way of the DSP algorithm to generate sound at a same frequency as the noise generated by the fan, but that is out of phase with the noise generated by the fan, so as to cancel out the noise generated by the fan and reduce the level of audible acoustic noise generated thereby.

5. The CT system of claim 1 further comprising:
an x-ray source heat exchanger configured to provide cooling to the x-ray source; and
a detector heat exchanger configured to provide cooling to the detector array;
wherein each of the x-ray source heat exchanger and the detector heat exchanger comprises:
a cooling unit configured to cool a cooling fluid and pump the cooling fluid through tubing;
a fan plenum;
a fan positioned within the fan plenum, the fan configured to either push air over or pull air away from the cooling unit so as to draw heat energy out from the cooling fluid; and
an outlet duct configured to discharge heated air out from the heat exchanger.

6. The CT system of claim 5 wherein the passive noise mitigation device comprises a layer of acoustic foam positioned within the outlet duct, the layer of acoustic foam configured to reduce the high frequency content of noise generated by the fan so as to reduce the level of audible acoustic noise generated thereby.

7. The CT system of claim 5 wherein the active noise mitigation device comprises:
a speaker positioned within the outlet duct;
a reference microphone positioned in proximity to the outlet duct to measure noise generated by the fan;
a controller configured to:
receive an output from the reference microphone indicative of the measured noise generated by the fan;
apply a digital signal processing (DSP) algorithm in order to determine a proper frequency and phase at which noise should be generated by the speaker, based on the measured noise; and
control the speaker by way of the DSP algorithm to generate sound at a same frequency as the noise generated by the fan, but that is out of phase with the noise generated by the fan, so as to cancel out the noise generated by the fan and reduce the level of audible acoustic noise generated thereby.

8. The CT system of claim 7 wherein the controller implements one of a feed-forward or feed-back control technique to control the speaker, with the controller receiving input from only the reference microphone when implementing the feed-forward control technique and the controller receiving input from the reference microphone and a separate error microphone when implementing the feedback control technique.

9. The CT system of claim 1 wherein the hybrid noise mitigation system is configured to reduce the level of audible acoustic noise generated by the CT system within the gantry opening and in an area surrounding the CT system.

10. The CT system of claim 1 wherein the outer housing substantially encloses the rotatable gantry so as to control noise generated by the CT system in a passive manner.

11. A computed tomography (CT) system comprising:
a rotatable gantry having a gantry opening to receive an object to be scanned;
an outer housing positioned about the rotatable gantry, the outer housing having gantry inlet ducts and gantry exhaust ducts formed therein each including a fan for transferring air into and out of an interior of the outer housing, respectively;
an x-ray source mounted on the rotatable gantry and configured to project an x-ray beam toward the object;
a detector array mounted on the rotatable gantry and configured to detect x-ray energy passing through the object and generate a detector output responsive thereto that can be reconstructed into an image of the object;
a heat exchanger corresponding to each of the x-ray source and the detector array and mounted on the rotatable gantry, the heat exchangers configured to provide cooling to the x-ray source and the detector array; and
a plurality of noise mitigation devices configured to mitigate noise generated by the CT system during operation thereof, wherein a noise mitigation device is provided for each of the gantry inlet ducts, gantry exhaust ducts, and heat exchangers to mitigate noise produced thereby in at least one of a passive manner and an active manner.

12. The CT system of claim 11 wherein the plurality of noise mitigation devices comprises a layer of acoustic foam positioned within the gantry inlet duct and the gantry exhaust duct, the layer of acoustic foam configured to reduce the high frequency content of noise generated by the fans therein so as to passively reduce a level of audible acoustic noise generated by the fans.

13. The CT system of claim 11 wherein the plurality of noise mitigation devices comprises an active noise mitigation device corresponding to each of the gantry inlet duct and the gantry exhaust duct, wherein each active noise mitigation device comprises:
a speaker positioned within the gantry inlet duct and the gantry exhaust duct;
a microphone positioned in proximity to the gantry inlet duct and the gantry exhaust duct to measure noise generated by the respective fans;
a controller configured to:
receive an output from the microphone indicative of the measured noise generated by the respective fan;
apply a digital signal processing (DSP) algorithm in order to determine a proper frequency and phase at which noise should be generated by the speaker, based on the measured noise; and
control the respective speaker by way of the DSP algorithm to generate sound at a same frequency as the noise generated by the respective fan, but that is out of phase with the noise generated by the fan, so as to cancel out the noise generated by the respective fan and actively reduce the level of audible acoustic noise generated thereby.

14. The CT system of claim 11 wherein the heat exchanger corresponding to each of the x-ray source and the detector array comprises:
a cooling unit configured to cool a cooling fluid and pump the cooling fluid through tubing;
a fan plenum;
a fan positioned within the fan plenum, the fan configured to either push air over or pull air away from the cooling unit so as to draw heat energy out from the cooling fluid; and
an outlet duct configured to discharge heated air out from the heat exchanger.

15. The CT system of claim 14 wherein the plurality of noise mitigation devices comprises a layer of acoustic foam positioned within the outlet duct, the layer of acoustic foam configured to reduce the high frequency content of noise generated by the fan so as to passively reduce a level of audible acoustic noise generated by the fan.

16. The CT system of claim 14 wherein the plurality of noise mitigation devices comprises an active noise mitigation device corresponding to each of the heat exchangers, wherein each active noise mitigation device comprises:
a speaker positioned within the outlet duct;
a microphone positioned adjacent the outlet duct to measure noise generated by the fan;
a controller configured to:
receive an output from the microphone indicative of the measured noise generated by the fan;
apply a digital signal processing (DSP) algorithm in order to determine a proper frequency and phase at which noise should be generated by the speaker, based on the measured noise; and
control the speaker by way of the DSP algorithm to generate sound at a same frequency as the noise generated by the fan, but that is out of phase with the noise generated by the fan, so as to cancel out the noise generated by the fan and actively reduce the level of audible acoustic noise generated thereby.

17. A method for mitigating noise in a computed tomography (CT) system comprising:
integrating a plurality of noise mitigation devices into existing components and features of the CT system;
passively reducing the level of audible acoustic noise generated by the CT system by way of the plurality of noise mitigation devices; and
actively reducing the level of audible acoustic noise generated by the CT system by way of the plurality of noise mitigation devices;
wherein the plurality of noise mitigation devices are configured to reduce the level of audible acoustic noise generated by at least one of CT gantry rotation, gantry fans, x-ray tube operation, x-ray tube heat exchanger fans, and x-ray detector heat exchanger fans.

18. The method of claim 17 wherein passively reducing the level of audible acoustic noise comprises integrating a layer of acoustic foam into each of each of a gantry housing inlet duct, a gantry housing exhaust duct, the x-ray source heat exchanger, and the detector heat exchanger, so as to mitigate noise generated by a fan included therein, the layer of acoustic foam configured to reduce the high frequency content of noise generated by the fans so as to passively reduce a level of audible acoustic noise generated by the fans.

19. The method of claim 17 wherein actively reducing the level of audible acoustic noise comprises controlling a speaker positioned in proximity to each of the gantry fans, x-ray tube heat exchanger fans, and x-ray detector heat exchanger fans, by way of a controller so as to generate sound at a same frequency as the noise generated by the respective fans, but that is out of phase with the noise generated by the respective fans, so as to cancel out the noise generated by the respective fans and actively reduce the level of audible acoustic noise generated thereby.

20. The method of claim 19 wherein controlling a respective speaker comprises controlling a respective speaker according to a feed-forward technique, the feed-forward technique comprising:
measuring noise generated by a respective fan by way of a reference microphone positioned in proximity thereto;
providing an output from the reference microphone indicative of the measured noise generated by the fan to the controller;
causing the controller to apply a digital signal processing (DSP) algorithm to the measured noise in order to determine a proper frequency and phase at which noise should be generated by the speaker; and
controlling the speaker by way of the DSP algorithm to generate sound at the same frequency as the noise generated by the fan, but out of phase with the noise generated by the fan, so as to cancel out the noise generated by the fan and actively reduce the level of audible acoustic noise generated thereby.

21. The method of claim 19 wherein controlling a respective speaker comprises controlling a respective speaker according to a feedback technique, the feedback technique comprising:
measuring noise generated by a respective fan by way of a reference microphone positioned in proximity thereto;
providing an output from the reference microphone indicative of the measured noise generated by the fan to the controller;
causing the controller to apply a digital signal processing (DSP) algorithm to the measured noise in order to determine a proper frequency and phase at which noise should be generated by the speaker;
controlling the speaker by way of the DSP algorithm to generate sound at the same frequency as the noise generated by the fan, but out of phase with the noise generated by the fan, so as to cancel out the noise generated by the fan and actively reduce the level of audible acoustic noise generated thereby;
measuring any acoustic noise present after generation of the speaker sound by way of an error microphone; and
providing an output from the error microphone to the controller to adjust the sound generated by the speaker, so as to further minimize an acoustic noise level.

22. The method of claim 19 wherein the controller comprises one of a component level controller and a CT system level controller.

23. The method of claim 17 wherein the plurality of noise mitigation devices are caused to actively reduce the level of audible acoustic noise generated by the CT system upon detection of a noise level rising above a noise level threshold.
